(19) United States
(12) Patent Application Publication
(10) Pub. No.: US 2021/0009785 A1
(43) Pub. Date: Jan. 14, 2021

(54) ACCELERATOR SOLUTIONS USEFUL FOR RESIN CURING

(71) Applicant: Arkema Inc., King of Prussia, PA (US)

(72) Inventors: Alaaeddin Alsbaiee, King of Prussia, PA (US); Evan Crocker, Downingtown, PA (US)

(21) Appl. No.: 16/969,578

(22) PCT Filed: Feb. 11, 2019
(86) PCT No.: PCT/US2019/017462

§ 371 Date: Aug. 13, 2020

Related U.S. Application Data
(60) Provisional application No. 62/631,997, filed on Feb. 19, 2018.

(51) Int. Cl. C08K 5/00; C08K 5/09; C08K 5/17; C08K 5/14; C08K 5/053; C08L 67/06

(52) U.S. Cl. CPC C08K 5/0091; C08K 5/0025; C08K 5/09; C08K 2201/019; C08K 5/14; C08K 5/053; C08L 67/06; C08K 5/17

(57) ABSTRACT

Accelerator solutions containing transition metal complexes based on organic ligands having one or more S—C—N, S—C—C—N, or S—C(═S)—S moieties are useful for accelerating the peroxide cure of resins such as unsaturated polyester resins.

### FIELD OF THE INVENTION

The present invention generally relates to accelerator solutions, methods for making such accelerator solutions, methods for curing curable resins using such accelerator solutions, cured resins obtained using such accelerator solutions, pre-accelerated curable resins containing such accelerator solutions, and two component systems in which such pre-accelerated curable resins comprise one component.

### BACKGROUND OF THE RELATED ART

Peroxides are commonly used as initiators to cure (crosslink) various types of resins, in particular resins containing monomers and/or oligomers having sites of ethylenic unsaturation such as unsaturated polyester resins and acrylic resins. A frequent practice in this art is to employ one or more metal-based catalysts or promoters in combination with peroxide to modify or control the curing characteristics of the peroxide.

Traditional methods to promote liquid organic peroxides (such as methyl ethyl ketone peroxide, also known as MEKP) involve the use of transition metal catalysts (such as cobalt salts) which react with peroxide via a redox mechanism. In this reaction, Co(II) ions oxidize to Co(III) and the peroxide initiator is reduced, generating reactive RO⋅ radicals which effectively initiate the polymerization of unsaturated polyester resins, acrylic resins and the like. Although cobalt salts have been the most widely used catalysts for promoting peroxides such as MEKP, such catalysts suffer from significant drawbacks such as 1) high toxicity to humans and the environment generally even in ppm quantities, leading to increasingly strict governmental regulations throughout the world, 2) relatively high cost compared to catalysts based on other transition metals, and 3) their tendency to impart an intense color (brown or black) to the cured resin, which limits the commercial applications of the final products.

These disadvantages of the traditional cobalt-based catalysts have led to research into the possibility of employing other types of transition metal catalysts that are based on more abundant transition metals such as Fe, Cu, Zn and Ni, as these metals are less expensive and less toxic than Co and also tend to yield less colored cured resins. However, these metals are inherently less reactive than cobalt and their salts cannot alone adequately promote MEKP and other liquid peroxides. Therefore, new promotion systems and formulations have recently been developed in which these metals, especially Fe and Cu, have been mixed with oxygen-, and/or nitrogen-, and/or phosphorous containing organic ligands. However, these ligands often need to be used in relatively large quantities and in combination with nitrogen bases, reducing agents, stabilizers and other component to achieve the desired reactivity, and gel and cure rates/kinetics.

Furthermore, the resulting formulations often suffer from low long-term stability due to the precipitation of metal over time, which leads to reduced performance after prolonged storage.

Accordingly, it would be desirable to develop new accelerator systems for peroxide promoters that are Co-free, more environmentally advantageous, and which do not possess the detrimental attributes mentioned above while achieving desired reactivity, gel and/or cure rates, and good shelf life.

### SUMMARY OF THE INVENTION

It has now been discovered that certain organic compounds containing both sulfur and nitrogen atoms or trithiocarbonate moieties can be effective promoters for transition metals such as Fe, Cu, Ni and Zn. Such organic compounds may function as ligands for the transition metal, thereby increasing the reactivity of the transition metal with respect to its ability to promote peroxides to initiate the cure of ethylenically unsaturated resin systems such as unsaturated polyester resins, wherein polymerization of such systems may be effectively initiated even at ambient (room) temperature. Effective organic ligands for this purpose were found to be those that contain one or more structural subunits (moieties) characterized by having a sulfur atom and a nitrogen atom separated by one or two carbon atoms (i.e., S—C—N and S—C—C—N moieties), such as mercaptopyridine, acetyl thiourea, trithiocyanuric acid, rhodanine, cysteamine, 2-imino-4-thiobiuret, 2-(butylamino)ethanethiol, 2-aminothiophenol, N-phenylthiourea and thiourea (wherein when complexed with the transition metal, such ligand may be in deprotonated form) and the like. Compounds containing at least one trithiocarbonate moiety (S—C(═S)—S) were also found to function as effective ligands for purposes of the present invention.

Such organic ligands can be relatively low in cost and may be readily available from commercial sources, and yet effectively activate transition metals such as Fe, Cu, Ni and Zn (which are also relatively low in cost and have fewer toxicity/environmental concerns as compared to the conventional Co-based accelerators) so that the metals promote peroxides such as methyl ethyl ketone peroxide at room temperature, leading to the cure of ethylenically unsaturated resins such as unsaturated polyester resins with desirably high exotherms. It has been surprisingly discovered that the cure kinetics exhibited by particular transition metal complexes of this type can be further fine-tuned or controlled by varying the organic ligand/transition metal ratio and/or by the addition of varying amounts of inexpensive organic acids or bases. In addition, the organic ligands are employed in relatively low amounts (typically, less than 0.4 wt % with respect to the weight of curable resin) and are capable of producing cured resins having low or no color, which is highly desirable in certain end-use applications where the appearance of an article manufactured from such cured resin is important.

Thus, according to certain aspects of the invention, an accelerator solution is provided which is comprised of:
a) at least one transition metal complex of at least one transition metal selected from the group consisting of Fe, Cu, Zn, Ni, Mn, Cr, Sn, Au, Pd and Pt and at least one organic ligand comprised of at least one S—C—N, S—C—C—N or S—C(═S)—S moiety; and
b) at least one solvent.

In other aspects, a method of curing a curable resin is provided, wherein the method comprises a step of combining the curable resin with at least one peroxide and at least one such accelerator solution. The present invention also pertains to a cured resin obtained by such method.

According to other aspects, the present invention also provides a process for preparing an accelerator solution, comprising reacting at least one transition metal salt comprised of at least one of Fe, Cu, Zn, Ni, Mn, Cr, Sn, Au, Pd and Pt with at least one organic ligand comprised of at least one S—C—N, S—C—C—N, or S—C(═S)—S moiety in an organic solvent to form at least one transition metal complex from the at least one transition metal salt and the at least one organic ligand. Additional aspects of the invention provide an accelerator solution obtained by such process.

A pre-accelerated curable resin comprised of at least one curable resin and at least one accelerator solution is provided in other aspects of the invention, wherein the accelerator solution is comprised of:
a) at least one transition metal complex of at least one transition metal selected from the group consisting of Fe, Cu, Zn, Ni, Mn, Cr, Sn, Au, Pd and Pt and at least one organic ligand comprised of at least one S—C—N, S—C—C—N, or S—C(═S)—S moiety; and
b) at least one solvent.

A two component system comprising a first component and a second component is provided in other aspects of the invention, wherein the first component comprises at least one pre-accelerated curable resin in accordance with such pre-accelerated curable resin and the second component comprises at least one peroxide.

Still further aspects of the invention pertain to a cured resin composition comprising a cured resin and at least one transition metal complex of at least one transition metal selected from the group consisting of Fe, Cu, Zn, Ni, Mn, Cr, Sn, Au, Pd and Pt and at least one organic ligand comprised of at least one S—C—N, S—C—C—N or S—C(═S)—S moiety.

### DETAILED DESCRIPTION OF CERTAIN EMBODIMENTS OF THE INVENTION

Transition Metals

The transition metal component of the transition metal complex may be one or more transition metals selected from of Fe, Cu, Zn, Ni, Mn, Cr, Sn, Au, Pd and Pt. According to particular embodiments, the transition metal(s) are selected from the group consisting of Fe, Cu, Zn and Ni, or the group consisting of Fe, Cu and Zn, or the group consisting of Fe and Cu. In some embodiments copper is most preferred. The accelerator solution may be formulated to be essentially free or entirely free of cobalt. For example, the accelerator solution may be comprised of less than 100 ppm, less than 50 ppm, less than 25 ppm, less than 10 ppm or less than 1 ppm Co. Two or more transition metals may be present in the transition metal complex and the accelerator solution may be comprised of two or more transition metal complexes.

The transition metal can be in any oxidation state, including both higher and lower oxidation states.

The transition metal complex may generally be prepared by reacting a transition metal compound which functions as a source of the transition metal in the complex with an organic compound that functions as a source of the organic ligand component of the transition metal complex. Suitable transition metal compounds for such purpose generally include halides, nitrates, sulfates, sulphonates, phosphates, oxides and carboxylates of the aforementioned transition metals. Examples of suitable halides include bromides and chlorides. Examples of suitable carboxylates include lactate, 2-ethyl hexanoate, acetate, propionate, butyrate, oxalate, laurate, oleate, linoleate, palmitate, stearate, acetyl acetonate, octanoate, nonanoate, heptanoate, neodecanoate and naphthenate.

The transition metal may be present in the accelerator solution, determined as metal, in an amount which provides (once the accelerator solution is formulated into a curable composition), for example, 0.5 to 2 mmol metal per kilogram of curable resin.

The concentration of transition metal in the resin system to be cured may be selected and adjusted as may be needed to achieve a particular desired curing profile, but typically is from 0.1 to 5 mmol metal per kilogram of the curable resin.

Organic Ligands

Ligands useful in the transition metal complexes employed in accordance with the present invention include organic compounds which are comprised of at least one S—C—N, S—C—C—N, or S—C(═S)—S(trithiocarbonate) moiety or two or more of such moieties. That is, suitable ligands may generally be characterized as compounds in which there is present a) a sulfur atom separated by one or two carbon atoms from a nitrogen atom or b) a trithiocarbonate group. Without wishing to be bound by theory, it is believed that such S—C—N or S—C(═S)—S moieties may permit the ligand in at least certain embodiments of the invention to function as a bidendate ligand, wherein both a sulfur atom and a nitrogen atom or two sulfur atoms bond to the same transition metal atom in the transition metal complex. The bonds may be covalent in nature, including coordinate covalent bonds. The sulfur and nitrogen atoms in the aforementioned moieties may be protonated (where a sulfur atom is present as a part of a thiol group or where a nitrogen atom is present as part of a primary or secondary amino group, for example), but in certain embodiments of the invention one or more of the sulfur and/or nitrogen atoms in the precursor compound used as a source of the organic ligand is initially protonated, but becomes deprotonated as a result of reaction with the transition metal compound used to form the transition metal complex.

According to certain aspects of the invention, the transition metal complex contains at least one organic ligand which is comprised of at least one moiety corresponding to Formula (I):

—X—C—C—SH (I)

wherein X is N, NH or NH₂ and —X—C—C— is aliphatic or part of a saturated, unsaturated or aromatic ring structure.

According to other aspects, the transition metal complex contains at least one organic ligand which is comprised of at least one moiety corresponding to Formula (II):

—X—C(═S)—X¹— (II)

wherein X is S, SH, N or NH, X¹ is NH or NH₂, and the —X—C—X¹— moiety is aliphatic or part of a saturated, unsaturated or aromatic ring structure.

According to other aspects, the transition metal complex contains at least one organic ligand which is comprised of at least one moiety corresponding to Formula (III):

—S—C(═S)—S (III).

In still further embodiments of the invention, the at least one organic ligand present in the transition metal complex is comprised of at least one moiety selected from the group consisting of H₂N—C—C—SH, —N—C—SH wherein N and C are part of an aromatic ring, H₂N—C(═S)—, —NH—C(═S)—, and —S—C(═S)—S—.

Preferred organic ligands suitable for use in the present invention include mercaptopyridine, acetyl thiourea, trithiocyanuric acid, rhodanine, cysteamine, 2-imino-4-thiobiuret, 2-(butylamino)ethanethiol (butyl cysteamine), bis(carboxymethyl)trithiocarbonate, and thiourea, and deprotonated derivatives thereof. In certain embodiments, more preferred organic ligands include 2-(butylamino)ethanethiol (butyl cysteamine), acetyl thiourea, bis(carboxymethyl)trithiocarbonate, rhodamine, cysteamine, or combinations thereof.

The concentration of organic ligand in the resin system to be cured may be selected and adjusted as may be needed to achieve a particular desired curing profile, but typically is from 0.01 to 0.5% by weight of the curable resin.

Preferred Transition Metal Complexes

The following transition metal complexes have been found to be particularly effective in accelerating the cure of curable resins, especially unsaturated polyester resins, using peroxides:

Fe, Cu and Zn complexed with mercaptopyridine and deprotonated derivatives thereof;
Fe, Cu and Zn complexed with acetyl thiourea and deprotonated derivatives thereof;
Cu and Fe complexed with trithiocyanuric acid and deprotonated derivatives thereof;
Cu and Fe complexed with rhodanine and deprotonated derivatives thereof;
Cu complexed with cysteamine and deprotonated derivatives thereof;
Cu complexed with 2-(butylamino)ethanethiol (butyl cysteamine or BuCysA) and deprotonated derivatives thereof;
Cu complexed with bis(carboxymethyl)trithiocarbonate and deprotonated derivatives thereof;
Cu complexed with 2-imino-4-thiobiuret and deprotonated derivatives thereof;
Zn complexed with thiourea and deprotonated derivatives thereof;
and combinations thereof.

Solvents

In addition to at least one transition metal complex, an accelerator solution in accordance with the present invention also contains one or more solvents, which are typically organic solvents. Such solvents typically assist in solubilizing the transition metal complex and/or providing a suitable liquid medium in which to react an organic ligand precursor and a transition metal compound to form the transition metal complex. Depending upon the solvent selected and the nature of the transition metal compound and the organic ligand precursor, the solvent may also participate in the complexation of the transition metal. That is, the transition metal complex may contain one or more solvent molecules or derivatives thereof as a ligand, in addition to the organic ligands described elsewhere herein.

The particular solvent or combination of solvents present in the accelerator solution is not believed to be particularly critical and any of a wide variety of solvents may be employed. For instance, in various embodiments of the invention the accelerator solution is comprised of at least one solvent is selected from the group consisting of alcohols, aliphatic hydrocarbons, aromatic hydrocarbons, aldehydes, ketones, ethers, esters, phosphates, phosphites, carboxylic acids, amides, sulfoxides (e.g., dimethylsulfoxide) and N-alkyl pyrrolidones (e.g., N-methyl and N-ethyl pyrrolidinone) and combinations thereof. In some embodiments, alcohols are preferred.

As used herein, the term “alcohols” refers to any organic compound containing one or more hydroxyl groups per molecule. In one embodiment of the invention, an aliphatic alcohol is used. In another embodiment, the accelerator solution is comprised of at least one aliphatic polyalcohol (i.e., an aliphatic alcohol containing two or more hydroxyl groups per molecule, such as a glycol). Examples of suitable alcohols are polyalcohols (including glycols) such as ethylene glycol, diethylene glycol, dipropylene glycol, polyethylene glycols, glycerol, ethoxylated glycerol, pentaerythritol and ethoxylated pentaerythritol, as well as mono-alkylethers thereof. Other types of suitable alcohols include, but are not limited to, isobutanol, pentanol, benzyl alcohol and fatty alcohols.

Specific examples of suitable phosphates and phosphites include are diethyl phosphate, dibutyl phosphate, tributyl phosphate, triethyl phosphate (TEP), dibutyl phosphite, and triethyl phosphate.

Examples of suitable aliphatic hydrocarbon solvents include white spirits, odourless mineral spirits (OMS) and paraffins.

Examples of suitable aromatic hydrocarbon solvents include naphthenes and mixtures of naphthenes and paraffins, 1,2-dioximes, N-methyl pyrrolidinone, N-ethyl pyrrolidinone; dimethyl formamide (DMF); dimethylsulfoxide (DMSO); 2,2,4-trimethylpentanediol diisobutyrate (TxIB);

Suitable esters include, but are not limited to, esters such as dibutyl maleate, dibutyl succinate, ethyl acetate, butyl acetate, mono- and diesters of ketoglutaric acid, pyruvates, esters of ascorbic acid such as ascorbic palmitate, diethyl malonate and succinates.

Suitable ketones include 1,2-diketones, in particular diacetyl and glyoxal.

The accelerator solution may optionally comprise water. If present, the water content of the accelerator solution may be at least 0.01 wt % or at least 0.1 wt %, for example. The water content is preferably not more than 50 wt %, more preferably not more than 40 wt %, more preferably not more than 20 wt %, even more preferably not more than 10 wt %, and most preferably not more than 5 wt %, all based on the total weight of the accelerator solution.

Other Components of Accelerator Solutions

In addition to transition metal complex and solvent, an accelerator solution in accordance with the present invention may contain one or more additional types of components. Such additional components may, for example, have the effect of further accelerating or modifying the curing characteristics of a curable resin when admixed with peroxide and the accelerator solution. These types of components may be referred to as co-agents, promoters, auxiliary accelerators or other such terms.

In one embodiment, the accelerator solution may be additionally comprised of one or more bases, in particular one or more bases, especially one or more organic bases such as organic amines or other nitrogen-containing organic compounds. Such bases are distinguished from the organic ligands present in the transition metal complexes of the accelerator solution, which may in some cases be considered bases due to the presence of amine functional groups, although in one embodiment of the invention excess (unreacted) organic ligand precursor may be present in the accelerator solution and function as a co-agent, promoter or auxiliary accelerator.

Suitable exemplary bases which may be present in the accelerator solution and the pre-accelerated curable resin include primary, secondary, and tertiary amines such as triethyl amine, dimethylaniline, diethylaniline, or N,N-dimethyl-p-toludine (DMPT), polyamines such as 1,2-(dimethyl amine)ethane, secondary amines such as diethyl amine, ethoxylated amines such as triethanol amine, dimethylamino ethanol, diethanolamine, or monoethanolamine, and aromatic amines such as pyridine or bipyridine. The base is preferably present in the accelerator solution in an amount of 5-50 wt %. In the pre-accelerator curable resin, it is preferably present in an amount of 0.5-10 g/kg resin.

Where the organic ligand is bis(carboxymethyl)trithiocarbonate, the use of one or more bases in combination with such organic ligand has been found to be particularly advantageous.

In other embodiments of the invention, the accelerator solution may be additionally comprised of one or more carboxylic acids. Particularly preferred are saturated carboxylic acids, especially relatively short-chain saturated carboxylic acids such as C1-C6 saturated carboxylic acids. The carboxylic acid may contain one or more carboxylic acid functional groups per molecule. Butyric acid is an example of a particularly preferred carboxylic acid additive in the accelerator solution. Incorporating a carboxylic acid such as butyric acid in the accelerator solution has been found to reduce the cure time and increase the exotherm temperature observed when the accelerator solution is used in combination with a peroxide to cure a curable resin such as an unsaturated polyester resin. The amount of carboxylic acid in the accelerator solution may be varied so as to achieve a particular cure profile that may be desired, but typical amounts may be from 1 to 10 wt % based on the weight of the accelerator solution.

According to certain embodiments of the invention, the organic ligand may contain one or more carboxylic acid groups. Bis(carboxymethyl)trithiocarbonate is an example of such an organic ligand.

Other types of promoters that may optionally be present in the accelerator solution include carboxylate salts of ammonium, alkali metals, and alkaline earth metals, halide salts of alkali metals and alkaline earth metals, and 1,3-diketones.

Examples of suitable halide salts of alkali metals and alkaline earth metals include, for examples chloride and bromide salts of potassium, sodium, lithium, calcium, barium and magnesium such as NaCl, LiCl, KCl, MgCl₂, CaCl₂ and BaCl₂.

Examples of suitable 1,3-diketones include acetyl acetone, benzoyl acetone, and dibenzoyl methane, and acetoacetamides and acetoacetates such as diethyl acetoacetamide, dimethyl acetoacetamide, dipropylacetoacetamide, dibutylacetoacetamide, methyl acetoacetate, ethyl acetoacetate, propyl acetoacetate, and butylacetoacetate.

Examples of suitable carboxylate salts of ammonium, alkali metals, and alkaline earth metals are the 2-ethyl hexanoates (i.e., octanoates), nonanoates, heptanoates, neodecanoates, and naphthenates. The preferred alkali metal is K. Potassium 2-ethylhexanoate is an example of a particular suitable carboxylate salt. The salts may be added to the accelerator solution or the resin as such, or they may be formed in situ. For example, alkali metal 2-ethyl hexanoates can be prepared in situ in the accelerator solution, after addition of an alkali metal hydroxide and 2-ethyl hexanoic acid to the accelerator solution.

In other embodiments of the invention, the accelerator solution may additionally contain one or more reducing agents. These include ascorbic acid (L-ascorbic acid and D-isoascorbic acid), oxalic acid, mercaptans, sugars (fructose, glucose, and others), aldehydes, sodium formaldehyde sulphoxylate (SFS), phosphines, phosphites, sulphites, sulphides, and their mixtures thereof. Reducing agents may present in an amount typically from 0.1 to 5 wt % based on the weight of the accelerator solution.

In other embodiments of the invention, the accelerator solution may additionally contain one or more radical deactivators. These include nitroxide radical deactivators such as TEMPO, 4H-TEMPO, SG-1, and their derivatives. Incorporating radical deactivators in the accelerator solution has been found to increase the cure time without affecting the exotherm temperature observed when the accelerator solution is used in combination with a peroxide to cure a curable resin such as an unsaturated polyester resin. Radical deactivators may be present in an amount typically from 0.01 to 1 wt % based on the weight of the accelerator solution.

Methods of Making Transition Metal Complexes and Accelerator Solutions

The accelerator solution can be prepared by simply mixing the ingredients, optionally with intermediate heating and/or mixing steps. The transition metal complex can be added as a pre-formed complex to the solution or can be formed in situ by combining the organic ligand and and a transition metal salt with the solvent(s), optionally followed by heating. The mixture is stirred with or without heating until the components are dissolved and a homogeneous solution is obtained. The weight or molar ratio of organic ligand to transition metal salt may be varied as desired depending upon the particular transition metal salt(s) and organic ligand(s) selected. In certain embodiments, for example, an excess of organic ligand relative to transition metal salt may be employed, while in other embodiments the transition metal salt is in excess relative to the organic ligand. The pre-accelerated curable resin can be prepared in various ways, including by mixing the individual ingredients with the curable resin and by mixing the curable resin with the accelerator solution according to the present invention.

Curable Resins

Suitable resins which may be cured using accelerator solutions according to the invention or which may be present in the pre-accelerated curable resin composition include, but are not limited to, alkyd resins, unsaturated polyester (UP) resins, vinyl ester resins, (meth)acrylate resins (sometimes referred to as acrylic resins), polyurethanes, epoxy resins, and mixtures thereof. Preferred resins include (meth)acrylate resins, UP resins and vinyl ester resins. In the context of the present application, the terms “unsaturated polyester resin” and “UP resin” refer to combinations of unsaturated polyester resin(s) and ethylenically unsaturated monomeric compound(s) such as styrene, which are typically used to lower the viscosity of the unsaturated polyester resin and as cross linker during polymerization. Unsaturated polyester resins are condensation polymers typically formed by the reaction of polyols (also known as polyhydric alcohols) with saturated and/or unsaturated dibasic acids. The term “(meth)acrylate resin” refers to combinations of acrylate and/or methacrylate resins and ethylenically unsaturated monomeric compounds. Such UP resins and acrylate resins are well known in the art and commercially available. Curing is generally started by either combining an accelerator solution according to the invention and the initiator(s) (peroxide) with the curable resin, or combining the peroxide(s) with the pre-accelerated curable resin.

Unsaturated polyester resins useful in this invention comprise reactive resins dissolved in a polymerizable monomer or mixture of monomers. These reactive resins are formed by condensing a saturated dicarboxylic acid or anhydride and an unsaturated dicarboxylic acid or anhydride with a dihydric alcohol. Examples of these polyester resins include the products of the reaction of a saturated dicarboxylic acid or anhydride (e.g., phthalic anhydride, isophthalic acid, tetrahydrophthalic anhydride, hexahydrophthalic anhydride, endomethylene tetrahydrophthalic anhydride, tetrachlorophthalic anhydride, hexachloroendomethylene tetrahydrophthalic acid, succinic acid, glutaric acid, adipic acid, pimelic acid, suberic acid, azelaic acid or sebacic acid) and an unsaturated dicarboxylic acid or anhydride (e.g., maleic anhydride, fumaric acid, chloromaleic acid, itaconic acid, citraconic acid or mesaconic acid) with a dihydric alcohol (e.g., ethylene glycol, propylene glycol, butylene glycol, diethylene glycol, triethylene glycol or neopentyl glycol). Small amounts of a polyhydric alcohol (e.g., glycerol, pentaerythritol, trimethylopropane or sorbital) may be used in combination with the glycol.

A three-dimensional structure may be produced by reacting the unsaturated polyester through the unsaturated acid component with an unsaturated monomer which is capable of reacting with the unsaturated polyester to form cross-linkages. Suitable unsaturated monomers include styrene, methylstyrene, dimethylstyrene, vinyltoluene, divinylbenzene, dichlorostyrene, methyl acrylate, ethyl acrylate, methylacrylate, diallyl phthalate, vinyl acetate, triallyl cyanurate, acrylonitrile, acrylamide and mixtures thereof. The relative amounts of the unsaturated polyester and the unsaturated monomer in the unsaturated polyester resin composition may be varied over a wide range. The unsaturated polyester resin compositions generally contain 20% to 80% by weight of the monomer, the monomer content preferably being in the range from 30% to 70% by weight.

Vinyl ester resins include resins prepared by esterification of epoxy resins with unsaturated carboxylic acids such as acrylic acid and methacrylic acid, with the resulting product then dissolved in a reactive solvent such as styrene (typically to a concentration of 35 to 45 percent by weight).

Acrylate resins include acrylates, methacrylates, diacrylates and dimethacrylates, higher functionality acrylates and methacrylates, including both monomers and oligomers, as well as combinations thereof.

Non-limiting examples of suitable ethylenically unsaturated monomeric compounds include styrene and styrene derivatives like α-methyl styrene, vinyl toluene, indene, divinyl benzene, vinyl pyrrolidone, vinyl siloxane, vinyl caprolactam, stilbene, but also diallyl phthalate, dibenzylidene acetone, allyl benzene, methyl methacrylate, methyl acrylate, (meth)acrylic acid, diacrylates, dimethacrylates, acrylamides; vinyl acetate, triallyl cyanurate, triallyl isocyanurate, allyl compounds (such as (di)ethylene glycol diallyl carbonate), chlorostyrene, tert-butyl styrene, tert-butylacrylate, butanediol dimethacrylate and mixtures thereof. Suitable examples of (meth)acrylate reactive diluents are PEG200 di(meth)acrylate, 1,4-butanediol di(meth)acrylate, 1,3-butanediol di(meth)acrylate, 2,3-butanedioldi(meth)acrylate, 1,6-hexanediol di(meth)acrylate and its isomers, diethyleneglycol di(meth)acrylate, triethyleneglycol di(meth)acrylate, glycerol di(meth)acrylate, trimethylolpropane di(meth)acrylate, neopentyl glycol di(meth)acrylate, dipropyleneglycol di(meth)acrylate, tripropyleneglycol di(meth)acrylate, PPG250 di(meth)acrylate, tricyclodecane dimethylol di(meth)acrylate, 1,10-decanediol di(meth)acrylate, tetraethylene glycol di(meth)acrylate, trimethylolpropanetri(meth)acrylate, glycidyl(meth)acrylate, (bis)maleimides, (bis)citraconimides, (bis)itaconimides, and mixtures thereof.

The amount of ethylenically unsaturated monomer in a pre-accelerated curable resin in accordance with the present invention is preferably at least 0.1 wt %, based on the weight of the curable resin component, more preferably at least 1 wt %, and most preferably at least 5 wt %. The amount of ethylenically unsaturated monomer is preferably not more than 50 wt %, more preferably not more than 40 wt %, and most preferably not more than 35 wt %.

If an accelerator solution is used for curing a curable resin or for preparing a pre-accelerated resin, the accelerator solution is generally employed in amounts of at least 0.01 wt %, preferably at least 0.1 wt %, and preferably not more than 5 wt %, more preferably not more than 3 wt % of the accelerator solution, based on the weight of the curable resin.

Peroxides

Peroxides suitable for curing the curable resin, in cooperation with an accelerator solution in accordance with the invention, and suitable for being present in the second component of the two component composition of the present invention include inorganic peroxides and organic peroxides, such as conventionally used ketone peroxides, peroxyesters, diaryl peroxides, dialkyl peroxides, and peroxydicarbonates, but also peroxycarbonates, peroxyketals, hydroperoxides, diacyl peroxides, and hydrogen peroxide. Preferred peroxides are organic hydroperoxides, ketone peroxides, peroxyesters, and peroxycarbonates. Even more preferred are hydroperoxides and ketone peroxides. Preferred hydroperoxides include cumyl hydroperoxide, 1,1,3,3-tetramethylbutyl hydroperoxide, tert-butyl hydroperoxide, isopropylcumyl hydroperoxide, tert-amyl hydroperoxide, 2,5-dimethylhexyl-2,5-dihydroperoxide, pinane hydroperoxide, para-menthane-hydroperoxide, terpene-hydroperoxide and pinene hydroperoxide. Preferred ketone peroxides include methyl ethyl ketone peroxide, methyl isopropyl ketone peroxide, methyl isobutyl ketone peroxide, cyclohexanone peroxide, and acetylacetone peroxide.

Mixtures of two or more peroxides can also be used. For example, a combination of a hydroperoxide or ketone peroxide with a peroxyester may be employed.

Methyl ethyl ketone peroxide, peroxyesters, and/or monoperoxydicarbonates are particularly preferred peroxides for use in the present invention.

The amount of peroxide to be used for curing the curable resin is preferably at least 0.1 per hundred resin (phr), more preferably at least 0.5 phr, and most preferably at least 1 phr. The amount of peroxide is preferably not more than 8 phr, more preferably not more than 5 phr, most preferably not more than 2 phr.

Other Components

The above-mentioned accelerator solutions, curable resins and peroxides can be combined with any of the other additives conventionally used in the peroxide-cured resin art, such as fillers, fibers, pigments, phlegmatizers, inhibitors (e.g., inhibitors of oxidative, thermal and/or ultraviolet degradation), lubricants, thixotropic agents, co-agents and promoters.

Examples of suitable phlegmatizers include hydrophilic esters and hydrocarbon solvents.

Examples of suitable fibers include glass fibers, carbon fibers, polymeric fibers (e.g., aramid fibers), natural fibers and the like and combinations thereof. The fibers may be in any suitable form, including in the form of mats, tows and other such forms known in the art.

Examples of suitable fillers include quartz, sand, silica, aluminum trihydroxide, magnesium hydroxide, chalk, calcium hydroxide, clays, carbon black, titanium dioxide and lime, as well as organic fillers such as thermoplastics and rubbers.

Resin Curing

Curing of a curable resin in accordance with the invention may generally be started by combining the accelerator solution, peroxide and curable resin.

The amount of accelerator solution relative to the amount of curable resin may be varied as may be desired or needed depending upon the particular transition metal(s), organic ligand(s) and curable resin(s) used as well as the particular curing characteristics (cure profile, including exotherm peak time) of the formulation and the properties of the cured resin which are sought. For example, the transition metal loading may be about 50 to about 200 ppm based on the weight of curable resin and the ligand loading may be about 1000 to about 5000 ppm based on the weight of curable resin.

As a result of the storage stability of the accelerator solution of the present invention, it is possible to pre-mix the curable resin and the accelerator solution days or weeks before the addition of the peroxide and, consequently, the start of the actual curing process. This allows the production and sale on a commercial scale of a curable resin composition which already contains an accelerator. Also contemplated by the present invention are two component systems comprising a first component and a second component, wherein the first component comprises at least one pre-accelerated curable resin (a combination of at least one curable resin and at least one accelerator solution in accordance with the present invention) and the second component comprises at least one peroxide. As used herein, the term “two component system” refers to systems where two components (A and B) are physically separated from each other (for instance, in separate cartridges, compartments, totes, drums or other containers), wherein components A and B are physically combined (admixed) at the time the system is to be used to form a cured resin.

The present invention may also be practiced by way of a three component system, wherein the curable resin, peroxide and accelerator solution are physically separated from each other until such time it is desired to produce a cured resin, where at such time the three components are mixed together and the mixture permitted to cure (polymerize) as a result of the chemical interaction between the curable resin, peroxide and accelerator solution.

The peroxide may be mixed with the pre-accelerated curable resin, added to a pre-mix of curable resin and accelerator solution, or pre-mixed with the resin after which accelerator solution is added. The resulting mixture is mixed and dispersed. The curing process can be carried out at any temperature from −15° C. up to 250° C., depending on the initiator system, the accelerator system, any compounds or substances which are present for the purpose of modifying the curing rate, and the curable resin composition to be cured. In one embodiment, it is carried out at ambient temperatures commonly used in applications such as hand lay-up, spray-up, filament winding, resin transfer molding, coating (e.g., gel coat and standard coatings), in-mold coatings, button production, centrifugal casting, corrugated sheets or flat panels, relining systems, kitchen sinks via pouring compounds, and so forth. However, the present invention can also be used in SMC, BMC, pultrusion techniques, and the like, for which temperatures up to 180° C., more preferably up to 150° C., most preferably up to 100° C., are used.

The cured resin can be subjected to a post-cure treatment to further optimize the hardness and/or other properties. Such post-cure treatment is generally performed at a temperature in the range of 40−180° C. for 30 min to 15 hours.

End Uses and Applications

The cured resins find use in various applications, including marine applications (including boats and marine sport products like boat parts), chemical anchoring, roofing, construction, relining, pipes and tanks, flooring, windmill blades, wall panels, composite rebars, laminates, composite articles (including fiber-reinforced composite articles), electrical and electronic devices, transportation such as truck and car parts, aerospace, vehicles and the like.

Exemplary Aspects of the Invention

Various exemplary aspects of the invention may be summarized as follows:

Aspect 1: An accelerator solution comprised of, consisting essentially of or consisting of:
a) at least one transition metal complex of at least one transition metal selected from the group consisting of Fe, Cu, Zn, Ni, Mn, Cr, Sn, Au, Pd and Pt and at least one organic ligand comprised of at least one S—C—N, S—C—C—N or S—C(═S)—S moiety; and
b) at least one solvent.

Aspect 2: The accelerator solution of Aspect 1, wherein the at least one transition metal is selected from the group consisting of Fe, Cu, Zn and Ni.

Aspect 3: The accelerator solution of Aspect 1 or 2, wherein the at least one organic ligand is comprised of at least one moiety selected from:
Formula (I): —X—C—C—SH, wherein X is N, NH or NH₂ and —X—C—C— is aliphatic or part of a saturated, unsaturated or aromatic ring structure;
Formula (II): —X—C(═S)—X¹—, wherein X is S, SH, N or NH, X¹ is NH or NH₂, and the —X—C—X1- moiety is aliphatic or part of a saturated, unsaturated or aromatic ring structure; or
Formula (III): —S—C(C═S)—S—.

Aspect 4: The accelerator solution of any of Aspects 1 to 3, wherein the at least one organic ligand is comprised of at least one moiety selected from the group consisting of H₂N—C—C—SH, —N—C—SH wherein N and C are part of an aromatic ring, H₂N—C(═S)—, and —NH—C(═S)—.

Aspect 5: The accelerator solution of any of Aspects 1 to 4, wherein the at least one ligand is selected from the group consisting of mercaptopyridine, acetyl thiourea, trithiocyanuric acid, rhodanine, cysteamine, 2-imino-4-thiobiuret, bis(carboxymethyl)trithiocarbonate and thiourea, and deprotonated derivatives thereof.

Aspect 6: The accelerator solution of any of Aspects 1 to 5, wherein the accelerator solution does not contain Co.

Aspect 7: The accelerator solution of any of Aspects 1 to 6, wherein the accelerator solution is additionally comprised of at least one base or at least one carboxylic acid.

Aspect 8: The accelerator solution of any of Aspects 1 to 7, wherein the accelerator solution is additionally comprised of at least one nitrogen-containing base or at least one saturated aliphatic carboxylic acid.

Aspect 9: The accelerator solution of any of Aspects 1 to 8, wherein the composition is additionally comprised of at least one ethoxylated amine or at least one C1-C6 aliphatic carboxylic acid.

Aspect 10: The accelerator solution of any of Aspects 1 to 9, wherein the complex has been prepared by a process comprising combining a transition metal salt with at least one organic ligand comprised of at least one S—C—N, S—C—C—N, or S—C(═S)—S moiety in the presence of at least one solvent.

Aspect 11: The accelerator solution of any of Aspects 1 to 10, wherein the complex has been prepared by a process comprising combining, in the presence of at least one solvent, a transition metal salt with at least one organic ligand comprised of at least one moiety selected from:
Formula (I): —X—C—C—SH, wherein X is N, NH or NH₂ and —X—C—C— is aliphatic or part of a saturated, unsaturated or aromatic ring structure;
Formula (II): —X—C(═S)—X¹—, wherein X is S, SH, N or NH, X1 is NH or NH₂, and the —X—C—X1- moiety is aliphatic or part of a saturated, unsaturated or aromatic ring structure; or
Formula (III): —S—C(═S)—S—.

Aspect 12: The accelerator solution of any of Aspects 1 to 11, wherein the at least one solvent is selected from the group consisting of alcohols, aliphatic hydrocarbons, aromatic hydrocarbons, aldehydes, ketones, ethers, esters, phosphates, carboxylic acids, amides, sulfoxides and N-alkyl pyrrolidones and combinations thereof.

Aspect 13: The accelerator solution of any of Aspects 1 to 12, wherein the at least one solvent is selected from the group consisting of aliphatic polyalcohols.

Aspect 14: The accelerator solution of any of Aspects 1 to 13, wherein the transition metal complex is selected from the group consisting of acetyl thiourea complexes of Fe; acetyl thiourea complexes of Cu; acetyl thiourea complexes of Zn; thiocyanuric acid complexes of Fe; thiocyanuric acid complexes of Cu; rhodanine complexes of Fe; rhodanine complexes of Cu; cysteamine complexes of Cu; 2-(butylamino)ethanethiol (butyl cysteamine or BuCysA) complexes of Cu, bis(carboxymethyl)trithiocarbonate (CMTTC) complexes of Cu, mercaptopyridine complexes of Cu, mercaptopyridine complexes of Zn, mercaptopyridine complexes of Fe and iminothioburet complexes of Cu.

Aspect 15: A method of curing a curable resin, comprising combining the curable resin with at least one peroxide and at least one accelerator solution in accordance with any of Aspects 1 to 14.

Aspect 16: The method of Aspect 15, wherein the at least one peroxide is selected from the group consisting of organic peroxides.

Aspect 17: The method of Aspect 15, wherein the at least one peroxide is selected from the group consisting of ketone peroxides, peroxyesters, diaryl peroxides, dialkyl peroxides, peroxydicarbonates, peroxycarbonates, peroxyketals, hydroperoxides, diacyl peroxides and hydrogen peroxides.

Aspect 18: The method of Aspect 15, wherein the at least one peroxide comprises at least one peroxide selected from the group consisting of ketone peroxides, peroxyesters and/or monoperoxydicarbonates.

Aspect 19: The method of Aspect 15, wherein the at least one peroxide comprises methyl ethyl ketone peroxide.

Aspect 20: The method of Aspect 15, wherein the at least one peroxide is liquid at 25° C.

Aspect 21: The method of any of Aspects 15 to 20, wherein the curable resin is selected from the group consisting of alkyd resins, unsaturated polyester resins, vinyl ester resins, and (meth)acrylate resins.

Aspect 22: The method of any of Aspects 15 to 21, wherein the curable resin is selected from the group consisting of unsaturated polyester resins.

Aspect 23: A cured resin obtained by the method of any of Aspects 15 to 22.

Aspect 24: A process for preparing an accelerator solution, comprising reacting at least one transition metal salt comprised of at least one of Fe, Cu, Zn, Ni, Mn, Cr, Sn, Au, Pd and Pt with at least one organic ligand comprised of at least one S—C—N, S—C—C—N or S—C(═S)—S moiety in an organic solvent to form at least one transition metal complex from the at least one transition metal salt and the at least one organic ligand.

Aspect 25: An accelerator solution obtained by the process of Aspect 24.

Aspect 26: A pre-accelerated curable resin comprised of at least one curable resin and at least one accelerator solution in accordance with any of Aspects 1 to 14.

Aspect 27: A two component system comprising a first component and a second component, wherein the first component comprises at least one pre-accelerated curable resin in accordance with Aspect 26 and the second component comprises at least one peroxide.

Aspect 28: A cured resin composition comprising a cured resin and at least one transition metal complex of at least one transition metal selected from the group consisting of Fe, Cu, Zn, Ni, Mn, Cr, Sn, Au, Pd and Pt and at least one organic ligand comprised of at least one S—C—N, S—C—C—N, or S—C(═S)—S moiety.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the accelerator solutions, pre-accelerated curable resins or two component systems or methods for making or using the accelerator solutions, pre-accelerated curable resins or two component systems. Additionally, in some embodiments, the invention can be construed as excluding any element or process step not specified herein.

Although the invention is illustrated and described herein with reference to specific embodiments, the invention is not intended to be limited to the details shown. Rather, various modifications may be made in the details within the scope and range of equivalents of the claims and without departing from the invention.

### EXAMPLES

General Experimental Method:

Accelerator solutions are initially prepared by mixing a metal salt (Fe sulfate, Cu acetate, or Zn 2-ethylhexanoate), an organic ligand, 1 g diethylene glycol, and other additives (if any). The mixture is stirred at 45° C. for 30 min and then cooled to room temperature. The accelerator solution is then transferred and mixed with 25 g of a UP resin (Aropol® 2036 C—Ashland) and 0.5 g (2 phr with respect to resin) of methyl ethyl ketone peroxide (MEKP) (Luperox® DDM-9 from Arkema Inc.) in a paper cup. The resulting mixture is then quickly transferred into a test tube, and a thermocouple (to allow monitoring temperature over time to generate a time-dependent temperature curve of polymerization) is placed in the center of the tube and the assembly is left to cure at ambient temperature. This experiment allows measuring exotherm peak time and temperature and the gelation time. The gelation time (gel time) is the elapsed time (in minutes) between the start of the experiment and the time at which the temperature reaches 5.6° C. above the room temperature.

### Example 1

This example includes accelerator solutions prepared using metal salts of FeSO₄ heptahydrate (Fe Sulf), Cu(II)acetate (Cu Ac₂), or Zn(II)2-ethylhexanoate (Zn hex₂) ca. 80% in mineral spirits, and one of the following organic ligands: acetylthiourea (AcTU), cysteamine (CysA), 2-(butylamino)ethanethiol (butyl cysteamine or BuCysA), rhodanine (RN), trithiocyanuric acid (TCA), 2-imino-4-thiobiuret (ITB), bis(carboxymethyl)trithiocarbonate (CMTTC), and thiourea (TU). In some of these experiments, diisopropylethyl amine (DIPEA) is added as a base in an amount that corresponds to 1 or 2 equivalents relative to the amount of the organic ligand. The amount of the metal salt is 0.014 g for Fe sulf, 0.009 g for Cu Ac₂, and 0.016 g for Zn hex₂, which correspond to 2 mmol metal per kilogram of curable resin. The amount of the sulfur-containing ligand is 0.1 g, which corresponds to 10 wt % with respect to accelerator solution and 0.4 wt % with respect to curable resin. The metal salt, ligand, and DIPEA (in certain entries) are mixed with 1 g diethylene glycol to prepare accelerator solutions that are then used to cure UP resin with MEKP initiator according to the procedure discussed in the “general experimental method” section above.

Results of these experiments are disclosed in Table 1. Control (comparative or “comp.”) experiments (entries 1-3), in which metal salts are examined alone, show that Fe, Cu and Zn metals alone (i.e., in the absence of organic ligand) are not capable of promoting MEKP to cure UP within 2 hours. Entries 4-17 show examples of metal/ligand combinations that surprisingly led to cured resin within 2 hours.

TABLE 1

| | | | Base | Gel | Peak | Peak | Cure |
|---|---|---|---|---|---|---|---|
| | Metal | | (DIPEA), | time | time | exotherm | w/in |
| Entry | salt | Ligand | g | (min) | (min) | (° C.) | 2 h? |
| 1 (comp) | Cu | — | — | — | — | — | No |
| | Ac₂ | | | | | | |
| 2 (comp) | Fe | — | — | — | — | — | No |
| | sulf | | | | | | |
| 3 (comp) | Zn | — | — | — | — | — | No |
| | hex₂ | | | | | | |
| 4 | Cu | AcTU | — | 0.6 | 3.2 | 127 | Yes |
| 5 | Ac₂ | CysA | — | 0.5 | 3.8 | 108 | Yes |
| 6 | | BuCysA | 0.025 | 4.2 | 11.5 | 104 | Yes |
| 7 | | ITB | — | 9.7 | 29.8 | 109 | Yes |
| 8 | | CMTTC | 0.11 | 5.5 | 15.0 | 99 | Yes |
| 9 | | RN | — | 9.5 | 42.0 | 70 | Yes |
| 10 | | RN | 0.12 | 2.2 | 11.2 | 97 | Yes |
| 11 | | TCA | — | 9.5 | 19.7 | 90 | Yes |
| 12 | Fe | TCA | — | 22.8 | 37.0 | 101 | Yes |
| 13 | sulf | TCA | 0.07 | 6.0 | 12.0 | 102 | Yes |
| 14 | | RN | — | 32.0 | 42.6 | 93 | Yes |
| 15 | Zn | AcTU | — | 0.8 | 4.7 | 56 | Yes |
| 16 | hex₂ | TCA | — | 24.8 | 37.0 | 35 | Yes |
| 17 | | TU | — | 0.8 | 3.7 | 37 | Yes |

### Example 2 (of the Invention)

This example includes accelerator solutions prepared using various combinations of Cu(II)acetate (Cu Ac₂), Cystamine (CysA), with or without diethanolamine (DEA), with or without butyric acid (BA). All these accelerator combinations were made with 1 g DEG as a solvent. The UP curing experiments with these accelerator solutions were done according to the procedure mentioned in the “general experimental method” section above. The results disclosed in Table 2 show that the different accelerator solutions obtained in this example are capable of promoting MEKP to cure UP resin with kinetics (i.e., rate of cure/gel time) which are controllable and have relatively high exotherm temperatures which are desirable because they indicate a relatively high degree of polymerization.

TABLE 2

| | CuAc₂ | CysA | DEA | BA | Gel | Peak | Peak |
|---|---|---|---|---|---|---|---|
| | (mMol/ | (wt % in | (wt % in | (wt % in | time | time | exotherm |
| Entry | kg resin) | resin) | resin) | resin) | (min) | (min) | (° C.) |
| 18 | 1.0 | 0.2 | — | — | 13.3 | 23.5 | 125 |
| 19 | 1.0 | 0.4 | — | — | 3.0 | 9.3 | 134 |
| 20 | 1.0 | 0.15 | — | 0.08 | 10.5 | 19.2 | 127 |
| 21 | 1.0 | 0.1 | — | 0.11 | 16.7 | 25.3 | 136 |
| 22 | 0.75 | 0.08 | 0.05 | — | 9.4 | 18.0 | 137 |
| 23 | 0.75 | 0.08 | 0.03 | — | 17.5 | 34.1 | 126 |

### Example 3 (of the Invention)

This example includes accelerator solutions prepared using various combinations of Cu(II)acetate (Cu Ac₂), Acetylthiourea (AcTU), with or without monoethanolamine (MEA), and with or without diethanolamine (DEA) as a base. All these accelerator combinations were made with 1 g DEG as a solvent. The UP curing experiments with these accelerator solutions were done according to the procedure mentioned in the “general experimental method” section above. The results are disclosed in Table 3.

TABLE 3

| | CuAc₂ | AcTU | DEA | MEA | Gel | Peak | Peak |
|---|---|---|---|---|---|---|---|
| | (mMol/ | (wt % in | (wt % in | (wt % in | time | time | exotherm |
| Entry | kg resin) | resin) | resin) | resin) | (min) | (min) | (° C.) |
| 24 | 1.0 | 0.4 | — | — | 0.5 | 8.0 | 109 |
| 25 | 0.75 | 0.075 | — | 0.15 | 1.7 | 11.3 | 130 |
| 26 | 1.0 | 0.08 | 0.2 | — | 1.7 | 7.2 | 137 |

### Example 4 (of the Invention)

This example includes accelerator solutions prepared using various combinations of Cu(II)acetate (CuAc₂), bis(carboxymethyl)trithiocarbonate (CMTTC), monoethanolamine (MEA), and diethanolamine (DEA). All these accelerator combinations were made with 1 g DEG as a solvent. The UP curing experiments with these accelerator solutions were done according to the procedure mentioned in the “general experimental method” section above. The results are disclosed in Table 4.

TABLE 4

| | CuAc₂ | CMTTC | DEA | MEA | Gel | Peak | Peak |
|---|---|---|---|---|---|---|---|
| | (mMol/kg | (wt % in | (wt % in | (wt % in | time | time | exotherm |
| Entry | resin) | resin) | resin) | resin) | (min) | (min) | (° C.) |
| 27 | 1.5 | 0.2 | 0.4 | — | 6.0 | 10.2 | 140 |
| 28 | 0.75 | 0.1 | 0.25 | — | 14.3 | 12.7 | 138 |
| 29 | 0.5 | 0.1 | 0.25 | — | 17.2 | 26.0 | 134 |
| 30 | 0.5 | 0.1 | — | 0.25 | 8.0 | 15.8 | 139 |
| 31 | 0.5 | 0.05 | — | 0.2 | 9.8 | 21.8 | 125 |

### Example 5 (of the Invention)

This example includes accelerator solutions prepared using various combinations of Cu(II)acetate (Cu Ac₂), Rhodanine (RN), monoethanolamine (MEA), and diethanolamine (DEA) as a base. All these accelerator combinations were made with 1 g DEG as a solvent. The UP curing experiments with these accelerator solutions were done according to the procedure mentioned in the “general experimental method” section above. The results are disclosed in Table 5.

TABLE 5

| | CuAc₂ | RN | DEA | MEA | Gel | Peak | Peak |
|---|---|---|---|---|---|---|---|
| | (mMol/ | (wt % in | (wt % in | (wt % in | time | time | exotherm |
| Entry | kg resin) | resin) | resin) | resin) | (min) | (min) | (° C.) |
| 32 | 1.0 | 0.1 | 0.6 | — | 3.2 | 6.5 | 153 |
| 33 | 0.75 | 0.075 | 0.4 | — | 6.0 | 11.0 | 145 |
| 34 | 0.5 | 0.05 | 0.25 | — | 8.0 | 14.8 | 136 |
| 35 | 0.3 | 0.05 | 0.25 | — | 11.3 | 18.3 | 133 |
| 36 | 0.3 | 0.05 | — | 0.25 | 5.7 | 10.5 | 151 |
| 37 | 0.3 | 0.03 | — | 0.25 | 6.2 | 13.5 | 135 |

### Example 6 (of the Invention)

This example includes accelerator solutions prepared using various combinations of Cu(II)acetate (Cu Ac₂), 2-(butylamino)ethanethiol (butyl cysteamine or BuCysA), monoethanolamine (MEA), and diethanolamine (DEA). All these accelerator combinations were made with 1 g DEG as a solvent. The UP curing experiments with these accelerator solutions were done according to the procedure mentioned in the “general experimental method” section above. The results are disclosed in Table 6.

TABLE 6

| | CuAc₂ | BuCysA | DEA | MEA | Gel | Peak | Peak |
|---|---|---|---|---|---|---|---|
| | (mMol / | (wt % in | (wt % in | (wt % in | time | time | exotherm |
| Entry | kg resin) | resin) | resin) | resin) | (min) | (min) | (° C.) |
| 38 | 2.0 | 04 | 0.1 | — | 1.8 | 6.3 | 125 |
| 39 | 1.0 | 0.1 | 0.1 | — | 19.3 | 38.5 | 119 |
| 40 | 1.0 | 0.1 | — | 0.2 | 9.0 | 17.8 | 141 |
| 41 | 0.5 | 0.05 | — | 0.3 | 11.3 | 24.0 | 137 |

### Example 7 (of the Invention)

This example demonstrates the ability of the accelerator system to promote peroxide families other than the methyl ethyl ketone peroxides. The accelerator solution for these tests were prepared using Cu(II)acetate (Cu Ace) at 0.7 mMol/kg resin, 2-(butylamino)ethanethiol (butyl cysteamine or BuCysA) at 0.063 wt % relative to the resin, monoethanolamine (MEA) at 0.3 wt % relative to the resin, and DEG as a solvent. The UP curing experiments with these accelerator solutions were done according to the procedure mentioned in the “general experimental method” section above. The results are disclosed in Table 7.

TABLE 7

| | | Peak | Peak |
|---|---|---|---|
| | | time | exotherm |
| Entry | Peroxide | (min) | (° C.) |
| 42 | Cumene hydroperoxide | 19.2 | 48 |
| 43 | T-butyl peroxybenzoate | 21.8 | 144 |
| 44 | OO-(t-Butyl) O-(2-Ethylhexyl) | 24.0 | 133 |
| | Monoperoxycarbonate | | |
| 45 | OO-(t-Amyl) O-(2-Ethylhexyl) | 18.5 | 132 |
| | Monoperoxycarbonate | | |
| 46 | Polyether Poly-t-butylperoxy | 20.2 | 139 |
| | Carbonate | | |
| 47 | 1,1-Di(t-butylperoxy)- | No cure | No cure |
| | cyclohexane | | |
| 48 | 1,1-Di(t-amylperoxy)- | No cure | No cure |
| | cyclohexane | | |

This table demonstrates that the accelerator system based on butyl cysteamine is able to cure hydroperoxides, peroxyesters and monoperoxydicarbonates at ambient temperature, but not peroxyketals.

1. An accelerator solution comprised of:
a) at least one transition metal complex of at least one transition metal selected from the group consisting of Fe, Cu, Zn, Ni, Mn, Cr, Sn, Au, Pd and Pt and at least one organic ligand comprised of at least one S—C—N, S—C—C—N or S—C(═S)—S moiety; and
a) at least one solvent.

2. The accelerator solution of claim 1, wherein the at least one transition metal is selected from the group consisting of Fe, Cu, Zn and Ni.

3. The accelerator solution of claim 1, wherein the at least one organic ligand is comprised of at least one moiety selected from:
Formula (I): —X—C—C—SH, wherein X is N, NH or NH₂ and —X—C—C— is aliphatic or part of a saturated, unsaturated or aromatic ring structure;
Formula (II): —X—C(═S)—X¹—, wherein X is S, SH, N or NH, X1 is NH or NH₂, and the —X—C—X1- moiety is aliphatic or part of a saturated, unsaturated or aromatic ring structure; or
Formula (III): —S—C(═S)—S—.

4. The accelerator solution of claim 1, wherein the at least one organic ligand is comprised of at least one moiety selected from the group consisting of H₂N—C—C—SH, —N—C—SH wherein N and C are part of an aromatic ring, H₂N—C(═S)—, and —NH—C(═S)—.

5. The accelerator solution of claim 1, wherein the at least one ligand is selected from the group consisting of mercaptopyridine, acetyl thiourea, trithiocyanuric acid, rhodanine, cysteamine, 2-imino-4-thiobiuret, thiourea, and bis(carboxymethyl)trithiocarbonate and deprotonated derivatives thereof.

6. The accelerator solution of claim 1, wherein the accelerator solution does not contain Co.

7. The accelerator solution of claim 1, wherein the accelerator solution is additionally comprised of at least one base or at least one carboxylic acid.

8. The accelerator solution of claim 1, wherein the accelerator solution is additionally comprised of at least one nitrogen-containing base or at least one saturated aliphatic carboxylic acid.

9. The accelerator solution of claim 1, wherein the composition is additionally comprised of at least one ethoxylated amine or at least one C1-C6 aliphatic carboxylic acid.

10. The accelerator solution of claim 1, wherein the complex has been prepared by combining a transition metal salt with at least one organic ligand comprised of at least one S—C—N, S—C—C—N or S—C(═S)—S moiety in the presence of at least one solvent.

11. The accelerator solution of claim 1, wherein the complex has been prepared by combining, in the presence of at least one solvent, a transition metal salt with at least one organic ligand comprised of at least one moiety selected from:
Formula (I): —X—C—C—SH, wherein X is N, NH or NH₂ and —X—C—C— is aliphatic or part of a saturated, unsaturated or aromatic ring structure;
Formula (II): —X—C(═S)—X¹—, wherein X is S, SH, N or NH, X1 is NH or NH₂, and the —X—C—X1- moiety is aliphatic or part of a saturated, unsaturated or aromatic ring structure; or
Formula (III): —S—C(═S)—S—.

12. The accelerator solution of claim 1, wherein the at least one solvent is selected from the group consisting of alcohols, aliphatic hydrocarbons, aromatic hydrocarbons, aldehydes, ketones, ethers, esters, phosphates, carboxylic acids, amides, sulfoxides and N-alkyl pyrrolidones and combinations thereof.

13. The accelerator solution of claim 1, wherein the at least one solvent is selected from the group consisting of aliphatic polyalcohols.

14. The accelerator solution of claim 1, wherein the transition metal complex is selected from the group consisting of complexes of acetyl thiourea with Fe; complexes of acetyl thiourea with Cu; complexes of acetyl thiourea with Zn; complexes of thiocyanuric acid with Fe; complexes of thiocyanuric acid with Cu; complexes of rhodanine with Fe; complexes of rhodanine with Cu; complexes of cysteamine with Cu; complexes of 2-(butylamino)ethanethiol (butyl cysteamine) with Cu; complexes of bis(carboxymethyl)trithiocarbonate with Cu; complexes of mercaptopyridine with Cu, mercaptopyridine complexes of Zn, mercaptopyridine complexes of Fe and complexes of iminothioburet with Cu.

15. A method of curing a curable resin, comprising combining the curable resin with at least one peroxide and at least one accelerator solution in accordance with claim 1.

16. The method of claim 15, wherein the at least one peroxide is selected from the group consisting of organic peroxides.

17. The method of claim 15, wherein the at least one peroxide is selected from the group consisting of ketone peroxides, peroxyesters, diaryl peroxides, dialkyl peroxides, peroxydicarbonates, peroxycarbonates, peroxyketals, hydroperoxides, diacyl peroxides and hydrogen peroxides.

18. The method of claim 15, wherein the at least one peroxide is selected from the group consisting of ketone peroxides, peroxyesters, monoperoxydicarbonates.

19. The method of claim 15, wherein the at least one peroxide comprises methyl ethyl ketone peroxide.

20. The method of claim 15, wherein the at least one peroxide is liquid at 25° C.

21. The method of claim 15, wherein the curable resin is selected from the group consisting of alkyd resins, unsaturated polyester resins, vinyl ester resins, and (meth)acrylate resins.

22. The method of claim 15, wherein the curable resin is selected from the group consisting of unsaturated polyester resins.

23. A cured resin obtained by the method of claim 15.

24. A process for preparing an accelerator solution, comprising reacting at least one transition metal salt comprised of at least one of Fe, Cu, Zn, Ni, Mn, Cr, Sn, Au, Pd and Pt with at least one organic ligand comprised of at least one S—C—N, S—C—C—N, or S—C(═S)—S moiety in an organic solvent to form at least one transition metal complex from the at least one transition metal salt and the at least one organic ligand.

25. An accelerator solution obtained by the process of claim 24.

26. A pre-accelerated curable resin comprised of at least one curable resin and at least one accelerator solution in accordance with claim 1.

27. A two component system comprising a first component and a second component, wherein the first component comprises at least one pre-accelerated curable resin in accordance with claim 26 and the second component comprises at least one peroxide.

28. A cured resin composition comprising a cured resin and at least one transition metal complex of at least one transition metal selected from the group consisting of Fe, Cu, Zn, Ni, Mn, Cr, Sn, Au, Pd and Pt and at least one organic ligand comprised of at least one S—C—N, S—C—C—N, or S—C(═S)—S moiety.
